(19) United States
(12) Patent Application Publication
(10) Pub. No.: US 2008/0152699 A1
(43) Pub. Date: Jun. 26, 2008

(54) Method of treating menopausal women transdermally with testosterone

(75) Inventors: Akshay Bhaskarrai Buch, West Chester, OH (US); Cynthia Anne Rodenberg, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/974,069

(22) Filed: Oct. 11, 2007

Related U.S. Application Data
(62) Division of application No. 11/606,815, filed on Nov. 30, 2006.
(60) Provisional application No. 60/741,160, filed on Dec. 1, 2005.

(51) Int. Cl. A61K 31/568; A61K 9/70; A61P 5/00

(52) U.S. Cl. 424/449; 514/170

(57) ABSTRACT

A method for administering testosterone to a menopausal woman in need of receiving transdermal testosterone therapy wherein the woman is being coadministered estrogen. The type and route of administration of the estrogen are determined and then changed according to the invention. Testosterone is transdermally administered to the woman. Also, a method for increasing the efficacy of testosterone administered to a menopausal woman in need of receiving transdermal testosterone therapy wherein the woman is being coadministered estrogen. Also, methods for the treatment of hypoactive sexual desire disorder in surgically menopausal and naturally menopausal women receiving concomitant estrogen. Also, methods for the treatment of vasomotor symptoms associated with menopause.

### CROSS REFERENCE TO RELATED APPLICATIONS

This application is a divisional of U.S. Ser. No. 11/606,815, (pending), filed on 30 Nov. 2006, which in turn claims priority (as does this application) from Provisional U.S. Ser. No. 60/741,160, filed 1 Dec. 2005, all of which are herein incorporated by reference in their entirety.

### FIELD OF THE INVENTION

The invention generally relates to female endocrinology, more specifically to transdermally administering testosterone to menopausal women who are receiving coadministered estrogen.

### BACKGROUND OF THE INVENTION

It is known to beneficially administer testosterone to menopausal women. One purpose for administering testosterone to women is in the treatment of Female Sexual Dysfunction (FSD). The main components of FSD are: Hypoactive Sexual Desire Disorder (HSDD), Sexual Arousal Disorder (FSAD), Orgasmic Disorder (FSOD), and Sexual Pain Disorder. HSDD is the persistent or recurring deficiency (or absence) of sexual fantasies, thoughts and/or desire for, or receptivity to, sexual activity, which causes personal distress. The cause may be either physiological or psychological or a combination of both. Common physiological etiologies include hormone deficiencies, medications, and surgical interventions. It has been reported in several national U.S. surveys that the proportion of menopausal women having low sexual desire (e.g. HSDD) ranges from 7-33%.

Testosterone may be transdermally administered to a woman. Such transdermal administration may be, e.g., via patch, gel, cream, spray, or ointment, or combinations thereof. Women in need of receiving transdermally administered testosterone may also be receiving estrogen replacement therapy (ERT). Some benefits of ERT are described in US Pat. App. No. 2002/0,173,499A1 (Pickar). Further benefits include treatment of moderate to severe vasomotor symptoms associated with menopause, atrophic vaginitis, osteoporosis, hypoestrogenism due to hypogonadism, castration, or primary ovarian failure, and breast cancer in selected persons with metastatic disease. Some indicators of improved health which may occur in women as a result of concomitantly receiving testosterone and oral estrogen supplementation are described in U.S. Pat. No. 6,853,129B1 (Mazer, et al.).

There exists a need to increase the efficacy of transdermally delivered testosterone in menopausal women who are being coadministered estrogen.

### SUMMARY OF THE INVENTION

A method for administering testosterone to a menopausal woman in need of receiving transdermal testosterone therapy wherein the woman is being coadministered estrogen. The type and route of administration of the estrogen are determined and then changed according to the invention. Testosterone is transdermally administered to the woman. Also, a method for increasing the efficacy of testosterone administered to a menopausal woman in need of receiving transdermal testosterone therapy wherein the woman is being coadministered estrogen. Also, methods for the treatment of hypoactive sexual desire disorder in surgically menopausal and naturally menopausal women receiving concomitant estrogen. Also, methods for the treatment of vasomotor symptoms associated with menopause.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a bar graph showing change from Baseline in 4-week Frequency of Satisfactory Sexual Events (SSE's) at Week 24.

FIG. 2 is a bar graph showing change from Baseline in Personal Distress Scale (PDS) at Week 24.

FIG. 3 is a bar graph showing change from Baseline in Sexual Desire at Week 24.

FIGS. 4A and 4B are pages 1 and 2, respectively, of a plot showing differences between Testosterone Transdermal System (TTS) and Placebo in mean change from Baseline for 4-week Frequency of Total Satisfying Episodes (TSE's) at Week 24 by Subgroup.

FIGS. 5A and 5B are pages 1 and 2, respectively, of a plot showing differences between TTS and Placebo in mean change from Baseline for Sexual Desire at Week 24 by Subgroup.

FIGS. 6A and 6B are pages 1 and 2, respectively, of a plot showing differences between TTS and Placebo in mean change from Baseline for frequency of TSE's at Week 24 by Subgroup.

### DETAILED DESCRIPTION OF THE INVENTION

Without being bound by theory, Applicants now believe that for women receiving transdermally administered testosterone coadministered with estrogen therapy, that women receiving conjugated estrogen exhibit lower efficacy of testosterone than women receiving non-conjugated estrogen. Again, without being bound by theory, Applicants now believe that a continuum exists where increasing efficacy is determined by type and route of administration of estrogen. In one aspect of the invention, this continuum is ordered: oral conjugated equine estrogen→oral conjugated non-equine estrogen→oral non-conjugated estrogen transdermal estrogen. In another aspect of the invention, this continuum is ordered: oral conjugated equine estrogen→oral conjugated non-equine estrogen→oral non-conjugated estrogen no estrogen.

In one aspect of the invention there is a new method of treating menopausal women with transdermal testosterone who are concomitantly receiving estrogen, which is believed to increase testosterone efficacy in the women. In another aspect, these women are suffering from FSD, HSDD, or both.

Without being bound by theory, Applicants now believe that women receiving conjugated estrogen have lower testosterone efficacy than women receiving oral non-conjugated estrogen or transdermal estrogen. Applicants believe that this reduced efficacy cannot be explained entirely by increased levels of sex hormone binding globulin (SHBG) in women receiving oral conjugated estrogen.

### DEFINITIONS

As used herein “coadministration” means the administration of multiple substances to one individual, either simultaneously or subsequently within the same therapeutic cycle. Thus, with reference to estrogen and testosterone, the term includes any situation in which women are receiving oral estrogen and non-oral testosterone. The term does not imply that the estrogen and testosterone have to be administered at the same time. Rather, as long as a woman is receiving oral estrogen, administration of non-oral testosterone will be within the present definition for coadministration. The skilled person will understand that the estrogen and the testosterone need not be provided in a single product or by an identical route to be coadministered. Coadministration is intended to be inclusive of the term “concomitant”.

As used herein “estrogen” means any substance, natural or synthetic, that exerts a biological or pharmacological action primarily by binding to estrogen receptors. Non-limiting examples include: 17β-estradiol and esters thereof including estradiol-3,17-diacetate, estradiol-3-acetate, estradiol-3,17-divalerate, estradiol-3-valerate, estradiol-17-valerate, and the corresponding pivalate, propionate, cypionate, benzoate, and like esters; ethinyl estradiol; 17-α-estradiol; estriol; and estrone. These estrogens may be derivatized or modified to form, e.g., conjugated equine estrogens, and esterified estrogens. Examples of esterified estrogens include but are not limited to: estradiol-3,17-diacetate, estradiol-3-acetate, estradiol-17-acetate, estradiol-3,17-divalerate, estradiol-3-valerate, estradiol-17-valerate. The estrogens may also be present as salts, (e.g., as sodium estrogen sulfate), isomers, or prodrugs. Also included, are phytoestrogens which are plant-derived estrogens. Isoflavones are one form of phytoestrogen and have a common diphenolic structure that resembles the structure of potent synthetic estrogens such as diethylstilbesterol and hexestrol. Exemplary isoflavones found in humans include, but are not limited to genistein, diadzein, and equol. Estrogen is intended to be inclusive of oral conjugated equine estrogen, oral conjugated non-equine estrogen, oral non-conjugated estrogen, and transdermal estrogen.

As used herein “oral estrogen” means any estrogen which is in a dosage form suitable for oral administration. Conjugated estrogens, conjugated equine estrogens, esterified estrogens and micronized estradiol are non-limiting examples of oral estrogens. Commercially available oral estrogen products include conjugated equine estrogens available under the trade name PREMARIN® from Wyeth-Ayerst Laboratories, esterified estrogens available under the trade name ESTRATAB® from Solvay Pharmaceuticals, and micronized 17-β-estradiol available under the trade name ESTRACE® from Bristol Meyers Squibb. Oral estrogen is intended to be inclusive of oral conjugated equine estrogen, oral conjugated non-equine estrogen, and oral non-conjugated estrogen.

As used herein “transdermal estrogen” means any estrogen which is in a dosage form suitable for transdermal administration.

As used herein “conjugated estrogen” means an estrogenic steroidal substance in which one or more functional groups (typically hydroxyl groups) on the steroid exists as a conjugate (typically a sulfate or glucuronide). The conjugated estrogens may be a single conjugated estrogen, or may consist of mixtures of various conjugated estrogens. Conjugated estrogens may also contain other steroidal or non-steroidal compounds, which may, or may not, contribute to the overall biological effect. Such compounds include, but are not limited to, unconjugated estrogens, androstanes, and pregnanes. Conjugated estrogen is intended to be inclusive of conjugated equine estrogen and conjugated non-equine estrogen. Examples of conjugated estrogens are PREMARIN (conjugated equine estrogens, USP) and CENESTIN (synthetic conjugated estrogens, A).

As used herein “conjugated equine estrogen” (CEE) means conjugated estrogen which contains a mixture of estrogens obtained from natural sources, blended to represent the average composition of material derived from pregnant mares' urine.

As used herein “conjugated non-equine estrogen” means conjugated estrogen that is not conjugated equine estrogen.

As used herein “non-conjugated estrogen” means estrogen which is not conjugated estrogen.

As used herein “oral conjugated equine estrogen” means conjugated equine estrogen which is in a dosage form suitable for oral administration.

As used herein “oral conjugated non-equine estrogen” means conjugated non-equine estrogen which is in a dosage form suitable for oral administration.

As used herein “oral non-conjugated estrogen” means non-conjugated estrogen which is in a dosage form suitable for oral administration.

As used herein “no estrogen” means that estrogen is not administered.

As used herein “testosterone” means the compound having the IUPAC names (17β)-17-Hydroxyandrost-4-en-3-one, and Δ⁴-androsten-17β-ol-3-one, as well as their isomers.

As used herein “sex hormone binding globulin”, or “SHBG”, also known as sex hormone binding protein (SHBP) and testosterone estradiol binding globulin (TeBG), means a serum protein that binds a variety of sex hormones with high affinity (See Dunn, et al., “Transport of Steroid Hormones: Binding of 21 Endogenous Steroids to Both Testosterone-Binding Globulin and Corticosteroid-Binding Globulin in Human Plasma”, Table 1, J. Clinical Endocrinology and Metabolism, Vol. 53:58-67 (1981)).

As used herein “free”, “total”, and “bioavailable” when used to refer to hormone serum levels, have the meaning set forth in U.S. Pat. No. 6,853,129B1 (Mazer, et al.).

The following two documents, NDA No. 21-769, “Briefing Document”, Procter & Gamble Pharmaceuticals, Inc., 2 Dec. 2004, and NDA No. 21-769, “Medical Review”, Procter & Gamble Pharmaceuticals, Inc., 2 Dec. 2004, both available at the FDA's website at URL: http://www.fda.gov/ohrms/dockets/ac/04/briefing/2004-4082b1.htm, are hereby incorporated by reference in their entirety.

Non-Limiting Examples of Hormones, Hormone-Containing Products, and Hormone Delivering Products

In one embodiment, a transdermal patch for administering testosterone to a woman is a matrix-type patch which comprises an occlusive backing that is impermeable to the testosterone and defines the face or top surface of the patch and a solid or semisolid matrix layer comprised of a homogeneous blend of the hormone, a polymeric pressure sensitive adhesive carrier, and optionally one or more skin permeation enhancers. Matrix patches are known in the art of transdermal drug delivery. Non-limiting examples of adhesive matrix transdermal patches are those described or referred to in U.S. Pat. Nos. 5,122,383 (Heiber, et al.) and 5,460,820 (Ebert, et al.).

In another embodiment, a transdermal patch for administering testosterone to a woman is a liquid reservoir system (LRS) type patch which comprises testosterone, and other optional ingredients, such as a permeation enhancer, in a carrier vehicle. The carrier vehicle comprises a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. LRS patches are known in the art of transdermal drug delivery. Non-limiting examples of LRS transdermal patches are those described or referred to in U.S. Pat. Nos. 4,849,224 (Chang, et al.) and 4,983,395 (Chang, et al.).

PREMARIN (conjugated estrogens tablets, USP) for oral administration contains a mixture of conjugated estrogens obtained from natural sources, occurring as the sodium salts of water-soluble estrogen sulfates blended to represent the average composition of material derived from pregnant mares' urine. It is a mixture of sodium estrone sulfate and sodium equilin sulfate, and at least the following 8 concomitant components, also as sodium sulfate conjugates: 17-α-dihydroequilin, 17-α-estradiol, Δ.8,9-dehydroestrone, 17-β-dihydroequilin, 17-β-estradiol, equilenin, 17-α-dihydroequilenin, and 17-β-dihydroequilenin.

CENESTIN (synthetic conjugated estrogens, A) tablets for oral administration contain a blend of 9 synthetic estrogenic substances: sodium estrone sulfate, sodium 17-α-dihydroequilin sulfate, sodium 17-α-estradiol sulfate, sodium equilenin sulfate, sodium 17-α-dihydroequilenin sulfate, sodium equilin sulfate, sodium 17-β-dihydroequilin sulfate, sodium 17-β-estradiol sulfate, sodium 17-α-dihydroequilenin sulfate.

In one embodiment, a synthetic conjugated estrogen comprises a mixture of estrogenic compounds, wherein the mixture comprises salts of conjugated estrone, conjugated equilin, conjugated Δ^{8,9}-dehyroestrone, conjugated 17-α-estradiol, conjugated 17-α-dihydroequilin, conjugated 17-β-dihydroequilin, conjugated 17-β-estradiol, conjugated equilenin, conjugated 17-α-dihydroequilin, and conjugated 17-β-dihydroequilenin, and wherein said mixture comprises the same essential estrogenic compounds present in naturally derived equine conjugated estrogens. In another embodiment, the mixture further comprises at least one compound selected from the group consisting of equilenin, 17-β-dihydroequilenin, 17-β-dihydroequilin, 17-α-dihydroequilenin, 17-β-estradiol, 17-α-dihydroequilin, 17-α-estradiol, equilin, Δ^{8,9}-dehydroestrone, estrone, 17-α-Δ^{8,9}-dehydroestradiol, 17-β-Δ^{8,9}-dehydroestradiol, 6-OH 17-α-dihydroequilenin, 6-OH equilenin, and the salts of 17-α-Δ^{8,9}-dehydroestradiol sulfate, 17-β-Δ^{8,9}-dehydroestradiol sulfate, 6-OH 17-α-dihydroequilenin sulfate, 6-OH 17-β-dihydroequilenin sulfate, and 6-OH equilenin sulfate. In yet another embodiment, the salts of 17-α-Δ^{8,9}-dehydroestradiol sulfate, 17-β-Δ^{8,9}-dehydroestradiol sulfate, 6-OH 17-α-dihydroequilenin sulfate and 6-OH equilenin sulfate are sodium salts. Such synthetic conjugated estrogens are described in U.S. Pat. No. 6,855,703B1 (Hill, et al.).

In one embodiment, CEE is characterized in that it comprises an alkali metal salt of 8,9-dehydroestrone-3-sulfate ester. In another embodiment, the salt is selected from sodium, potassium, and lithium. In another embodiment, the CEE is characterized in that it comprises an alkali metal salt of 8,9-dehyrdroestrone-3-sulfate ester in combination with tris(hydroxymethyl)aminomethane. Such CEE embodiments are described, e.g. in U.S. Pat. No. 5,210,081 (Raveendranath, et al.).

The following Table 1 contains a list of some of the estrogen preparations currently available in the United States and Europe. Listings of such preparations are available in such as the Physicians' Desk Reference, The FDA Orange Book, and the European equivalents thereof.

TABLE 1
Estrogen replacement therapies available in
the United States and/or Europe:

| Generic Name | Brand Name | Strength |
|---|---|---|
| Oral estrogens | | |
| Conjugated equine estrogens (natural) | PREMARIN | 0.3, 0.625, 0.9, 1.25, 2.5 mg |
| Conjugated estrogens (synthetic) | CENESTIN | 0.625, 0.9 mg |
| Esterified estrogens (75-80% estrone | ESTRATAB | 0.3, 0.625, 1.25, 2.5 mg |
| sulfate, 6-15% equilin sulfate derived | | |
| from plant sterols) | | |
| Estropipate (Piperazine estrone | OGEN ORTHO-EST | 0.625, 1.25, 2.5 mg |
| sulfate) | | |
| Micronized estradiol | ESTRACE | 0.5, 1.0, 2.0 mg |
| Esterified estrogens and | ESTRATEST | 1.25 mg esterified estrogen |
| methyltestosterone | | and 2.5 mg |
| | | methyltestosterone |
| | ESTRATEST HS | 0.625 mg esterified estrogen |
| | | and 1.25 mg methyltestosterone |
| Estradiol valerate | CLIMAVAL | 1 mg, 2 mg |
| Estradiol | ELLESTE SOLO | 1 mg, 2 mg |
| Estradiol | ESTROFEM | 2 mg |
| Estradiol | ESTROFEM FORTE | 4 mg |
| Piperazine esterone sulfate | HARMOGEN | 1.5 mg |
| Combination Products: | | |
| Estrone | HORMONIN | 1.4 mg |
| Estradiol | | 0.6 mg |
| Estriol | | 0.27 mg |
| Estradiol valerate | PROGYNOVA | 1 mg, 2 mg |
| Estradiol | ZUMENON | 1 mg, 2 mg |
| Transdermal estrogens: | | |
| Estradiol | ALORA (twice | 0.025, 0.0375, 0.05, 0.075, |
| | weekly) | 0.1 mg of estradiol released |
| | CLIMARA (weekly) | daily (dose options for |
| | ESTRADERM (twice | various products) |
| | weekly) | |
| | FEM PATCH (weekly) | |
| | VIVELLE (twice | |
| | weekly) | |
| Estradiol | DERMESTRIL | 25, 50, 100 .mu. g |
| Estradiol | ESTRADERM | 25, 50, 100 .mu. g |
| Estradiol | EVOREL (SYSTEN) | 25, 50, 75, 100 .mu. g |
| Estradiol | FEMATRIX | 40, 80 .mu. g |
| Estradiol | MENOREST | 25, 37.5, 50, 75 .mu. g |
| | PROGYNOVA TS | |
| Estradiol | TS FORTE | 50, 100 μg |
| | (CLIMARA) | |
| Vaginal estrogens: | | |
| Conjugated equine estrogens | PREMARIN vaginal | 0.625 mg/g |
| | cream | |
| Dienestrol | ORTHO | 0.1 mg/g |
| | DIENESTROL cream | |
| Estradiol | ESTRING | 7.5 μg |
| Estropipate | OGEN vaginal cream | 1.5 mg/g |
| Micronized estradiol | ESTRACE vaginal | 1.0 mg/g |
| | cream | |

Clinical Efficacy Studies

Clinical studies to evaluate the efficacy of a testosterone transdermal system (TTS) in the treatment of HSDD in surgically menopausal (SM) women consist of several trials (see Table 2). The two Phase IIb and the two Phase III trials are similar in design and use three instruments developed by Procter & Gamble Pharmaceuticals, Inc., in surgically menopausal women. These instruments are used to measure important efficacy measures, namely: the Sexual Activity Log (SAL) that captures the number of sexual episodes, the Profile of Female Sexual Function (PFSF) that measures desire and other aspects of sexuality, and the Personal Distress Scale (PDS) that measures distress associated with low sexual desire. The two blinded, placebo-controlled Phase III studies (2001133 and 2001134) are designed to evaluate efficacy.

TABLE 2
List of Controlled, Blinded Efficacy Studies

| | N @ | | |
|---|---|---|---|
| Study | Testosterone | N @ Placebo | |
| Phase/# | dose (mcg/day) | dose | Estrogen Therapy |
| II a T96006 | N = 75 @ 150 | N = 75 | Oral CEE |
| | N = 75 @ 300 | | |
| II b 1999068 | N = 107 @ 150 | N = 119 | Oral only |
| | N = 110 @ 300 | | |
| | N = 111 @ 450 | | |
| II b 1999092 | N = 37 @ 300 | N = 39 | Transdermal only |
| III 2001133 | N = 283 @ 300 | N = 279 | Oral and transdermal |
| III 2001134 | N = 266 @ 300 | N = 266 | Oral and transdermal |

Study 1999068 is a randomized, double blind, placebo-controlled multicenter trial investigating the effects of three TTS doses on sexual outcome measures using the SAL and PFSF. The 447 subjects are all hysterectomized and oophorectomized and have low libido. The study consists of an 8-week pre-treatment period followed by a 24-week efficacy period. Routine laboratory and testosterone/estrogen hormonal levels are followed from baseline through the 24 weeks.

Study 1999092 is a randomized, double blind, placebo-controlled multicenter trial investigating the effects of a 300 mcg/day dose on sexual outcome measures using the SAL and PFSF. The 77 subjects are all hysterectomized and oophorectomized, have low libido, and are on transdermal estrogen therapy (ET). The study consists of an 8-week pre-treatment period followed by a 24-week efficacy period. Subjects are stratified into 2 groups prior to randomization based on their dose of ET (≦0.05 mg, >0.05 mg). Routine laboratory and testosterone/estrogen hormonal levels are followed from baseline through the 24 weeks.

The two Phase 3 studies are nearly identical in design (clinical laboratory tests differ slightly between the studies). Each is a multicenter, multinational study conducted in SM women with HSDD. Each is based on a 24-week, randomized, double-blind (DB), placebo-controlled (PC), parallel-group design to assess efficacy.

Subjects are stratified based on their use of oral or transdermal ET. Within each stratum, subjects are randomly assigned in a 1:1 ratio to receive 300 mcg/day TTS or a placebo patch. Subjects apply the investigational TTS to their abdomen twice weekly (each patch is worn for approximately 3-4 days). Upon completion of the 24-week DB efficacy period of the study, subjects receiving placebo are switched to 300 mcg/day TTS, while the active cohort remained on active treatment. The subjects and study site personnel remain blinded to the initial randomized treatment throughout the open label portion of each study.

The primary objective of each study is to assess the efficacy of 300 mcg/day TTS in treating HSDD in SM women on concomitant oral or transdermal estrogen therapy. Efficacy is assessed by the change in the 4-week frequency of total satisfying sexual events (SSEs) from baseline to Week 24. Key secondary objectives are: personal distress as measured by the PDS score, and sexual desire as measured by the PFSF.

The frequency of satisfactory sexual events is recorded on the SAL that is completed by subjects at home on a weekly basis and returned to the clinical sites at the next scheduled visit. The PFSF and PDS are completed at the clinical sites at baseline (Week 0) and Weeks 4, 8, 12, and 24. Serum samples are analyzed in each study at baseline and Weeks 12 and 24 for determination of free, total, and bioavailable testosterone, SHBG, free and total estradiol, and estrone.

Inclusion criteria are developed to ensure that subjects entering the study are surgically menopausal, satisfy major diagnostic criteria for acquired (not chronic) HSDD, and are in stable relationships in which partner factors would not preclude the possibility of demonstrating a treatment effect.

Women are screened for study participation according to the following inclusion criteria at Week −8 (Visit 1) unless otherwise specified. Eligible women must: (a) be 20 to 70 years old and in generally good health based on medical history, physical examination, and laboratory evaluation; (b) undergo bilateral salpingo-oophorectomy and hysterectomy at least 6 months prior to screening and have no physical impediment to sexual function for at least 3 months prior to screening (documented evidence of surgery had to be provided). If the surgical report is not available, a history consistent with removal of both ovaries is to be obtained. Attempts to obtain the surgical report are documented; (c) be receiving a stable dose of ET for at least 3 months prior to screening with the intention of maintaining that regimen (i) This treatment should result in, on average, less than 3 moderate to severe hot flashes per day. (ii) Acceptable therapies include approved oral estrogens and approved transdermal estrogen patch regimens. (iii) Compounded estrogen preparations, selective estrogen receptor modulators (SERMs), or phytoestrogens preparations are not to be substituted for the ET requirement; (d) be, in her own judgment, in a stable monogamous sexual relationship that is perceived to be secure and communicative, for at least 1 year prior to study entry. The relationship is to be with the same partner who is sexually functional, both psychologically and physically, and expected to be physically present (i.e., available for sexual activity at some time during a 24-hour day) at least 50% of each month during the 8-week pretreatment and 24-week efficacy period of the study; (e) answer affirmatively to ALL five of the following questions: (i) Before your ovaries were removed, would you say that in general, your sex life is good and satisfying? (ii) Since your ovaries were removed, do you feel you have experienced a meaningful loss in your level of desire for sex? (iii) Since your ovaries were removed, do you feel you have experienced a significant decrease in your sexual activity? (iv) Are you concerned about or bothered by your current level of desire for or interest in sex? (v) Would you like to see an increase in your level of interest in or desire for sex and sexual activity? (f) if ≧40 years of age, have a clinically acceptable screening bilateral mammogram (no evidence of malignancy) as determined by the local radiologist (subjects could submit a mammogram done up to 2 months prior to screening). Subjects under 40 years of age at screening could elect to have mammograms if they wish, but these are not required for study entry; (g) have a clinically acceptable Pap smear as read by a central cytology facility (no evidence of malignancy or squamous intraepithelial lesions) if the cervix is present. A Pap smear at study entry could be performed on subjects without a cervix at the discretion of the investigator.

Exclusion criteria are developed to ensure that psychological or physical factors, other than low testosterone, that could cause hypoactive sexual desire are not present. Potential study subjects with medical conditions that might cause them to be at increased risk for adverse events (AEs) are also excluded, as are subjects taking drugs or nutritional supplements that are likely to affect sexual function.

Women are screened for study participation according to the following exclusion criteria at Week −8 (Visit 1) or as specified. Eligible women must not: (a) have dyspareunia not alleviated by lubricants, any physical limitations, or sexual trauma since their oophorectomy that would interfere with normal sexual function; (b) be experiencing any chronic or acute life stress relating to any major life change, such as recent loss of income or the death of a close family member, that could, in the opinion of the investigator, significantly interfered with sexual activity; (c) have significant psychiatric disorder (including mild depressive disorder, as evidenced by a score of ≧14 on the Beck Depression Inventory-II [BDI-II]), or have a significant alcohol or drug dependency and/or be receiving pharmacologic treatment for such illness or disorder; (d) have evidence of clinically significant organic disease on the history and/or physical examination that would, in the opinion of the investigator, prevent the patient from completing the study, or otherwise affect the outcome of the study. Subjects can not have any major illness, active gallbladder disease, gynecological or breast surgery, including excisional biopsies, within the last 6 months; (e) use within the last 12 weeks any medications/preparations that could affect sexual function or otherwise interfere with interpretation of study results. (f) use tablet or powder forms of phytoestrogens for less than 12 weeks prior to Week −8 (Visit 1). No use or stable use of phytoestrogens for 12 weeks or longer is acceptable; (g) use (averaging more than once a week) in the past 30 days the following preparations: dehydroepiandrosterone (DHEA), melatonin, or other drugs or supplements that could, in the opinion of the investigator, affect sexual function; (h) receive marketed or investigational oral, sublingual, topical, transdermal, injectable, or vaginal androgen therapy at any time during the past 3 months, or investigational implantable androgen therapy at any time in the past 7 months; (i) have current severe dermatological problems (e.g., severe or cystic acne) or have a known or suspected hypersensitivity or allergy to any adhesive or any of the constituents of the transdermal systems; (j) have a history of breast cancer or estrogen-dependent neoplasia (e.g., endometrial cancer) or any gynecological cancer at any time before study entry or other cancer (except basal or squamous cell carcinoma) within the last 5 years; (k) have diabetes, a history of cerebrovascular disease, thromboembolic disorders, myocardial infarction, or angina at any time before study entry or thrombophlebitis within the last 5 years; (l) have a thyroid-stimulating hormone (TSH) value at screening above the upper limit of normal confirmed by a free T4 outside the normal laboratory range (subjects with an abnormal TSH, normal free T4, and no clinical signs or symptoms of thyroid disease, with or without replacement treatment, could be admitted to the study); (m) have, in the opinion of the investigator, clinically significant abnormal pretreatment laboratory parameters (a single repeat assay is allowed if an assay result is questionable) that would materially impact the patient's ability to participate in the study; (n) have the following laboratory values: serum creatinine>2.5 mg/dL, serum total bilirubin≧2 times the upper limit of normal, alanine aminotransferase (ALT) or aspartate aminotransferase (AST)>3 times the upper limit of normal; or (o) previously participate in Study 1999068 or 1999092 or participate in a clinical trial within 30 days or received an investigational medication within 30 days (women participating in observational studies, however, could be included).

The Schedule of Visit Procedures for the Phase 3 clinical trials is listed in Table 3.

TABLE 3
Schedule of Visit Procedures: Phase 3 Trials

| | Screening | | | | | | | | |
|---|---|---|---|---|---|---|---|---|---|
| | Period | | Efficacy Treatment Period | | | | | | |
| | Visit 1 | Visit 2 | Visit 3 | Visit 4 | Visit 5 | Visit 6 | Visit 7 | Visit 8 | Visit 9 |
| PROCEDURE | Wk −8 | Wk −4 | Wk 0 | Wk 4 | Wk 8 | Wk 12 | Wk 16 | Wk 20 | Wk 24 |
| Informed Consent | X | | | | | | | | |
| Eligibility Criteria | X | X | X | | | | | | |
| Personal/Demographic Data | X | | | | | | | | |
| Medical, Gynecological and Drug History | X | X | X | | | | | | |
| Physical Examination (including breast and pelvic | | X | | | | | | | X |
| exam) | | | | | | | | | |
| Bilateral Mammogram | | X | | | | | | | |
| Vital signs (BP, heart rate, temperature), weight | X | | X | | | X | | | X |
| Cholesterol, triglycerides, LDL, HDL, glucose, | X | | | | | | | | X |
| hemoglobin A1c, insulin, serum chemistry, liver function tests, | | | | | | | | | |
| hematology | | | | | | | | | |
| Hormone measurements (Androgens, Estrogens and | | X | | | | X | | | X |
| SHBG) | | | | | | | | | |
| Hirsutism, Frequency of facial depilation, Acne score | | X | | | | X | | | X |
| Randomize to treatment | | | X | | | | | | |
| Review of inventories with patients | X | X | | | | | | | |
| Collect completed SAL | | X | X | X | X | X | X | X | X |
| Complete PFSF, PDS | | | X | X | X | X | | | X |
| Concomitant Medications | | | | X | X | X | X | X | X |
| AE Adverse Events | X | X | X | X | X | X | X | X | X |

The primary efficacy endpoint is the change from baseline in the total frequency of satisfying sexual events (SEE's) at 24 weeks (sum of the 4 consecutive weekly frequencies on SAL questions 3 and 6 during Weeks 21-24). Herein, the frequency of satisfying sexual events is presented as the total count over a 4-week period.

Secondary endpoints are the sexual desire domain score of the PFSF and the PDS Score (Distress) at 24 weeks. Each item within a domain has 6 possible answers ranging from all of the time to none of the time. Raw domain scores are computed by summing the scores for items within a domain and transforming the score to a 0 to 100 scale.

The primary analysis is based on the intent-to-treat (ITT) population.

The change from baseline in the total frequency of satisfying sexual activity, summed over Weeks 21-24, is regressed on treatment group, pooled center, baseline average 4 week frequency of satisfying sexual activity, age, and ET route in an ANCOVA analysis. Least squares estimates of the mean 4 week frequency of satisfying sexual activity for the 300 mcg/day testosterone arm and the placebo arm are computed, as are 95% confidence intervals and a p-value testing the null hypothesis of no treatment differences. Secondary models, including possible interactions (e.g., pooled center by treatment), are explored to evaluate consistency of treatment effects across other factors.

To account for subjects who did not complete the 24-week efficacy period of the study and therefore had missing SAL weekly frequencies for Weeks 21-24, a last observation carried forward (LOCF) approach is used. For each patient, the sexual activity corresponding to the last SAL and 3 preceding SALs is used in the analysis. For example, if the last SAL returned from a patient is the Week 23 SAL, then the frequency of sexual activity recorded for weeks 20-23 is used in the analysis. To account for missing weekly assessments within any four-week interval of follow-up, the average of non-missing assessments is imputed for the missing assessment.

The prospectively defined analyses of the primary endpoint are not the same in all studies. The Phase 2b studies used Poisson regression analysis, and the Phase 3 studies used ANCOVA and Wilcox-rank sums (WRS). The WRS is used in the Phase 3 studies to provide appropriate comparisons between 300 mcg/day TTS and placebo in cases where the ANCOVA assumptions are violated. Because a few influential outliers occur in Study 2001134, WRS is conducted for cross-study comparisons, in addition to ANCOVA. All efficacy analyses are based on the change from baseline in the total satisfying sexual activity at Week 24, using the ITT population and a LOCF approach.

Phase 3 Study 2001133 enrolls 562 subjects. A total of 451 (80%) subjects complete the efficacy period through Week 24. Of these subjects, 449 participate in the 28-week open label period and 380 (85%) completed through Week 52. Phase 3 Study 2001134 enrolls 533 subjects. A total of 418 (78%) subjects complete through Week 24. Of these subjects, 388 (93%) participate in the 28-week open label period and 261 (67%) of the 388 complete through Week 52 (see Table 4).

TABLE 4
Phase 3 Trial Enrollment and Completion

| | Study | | Study | | |
|---|---|---|---|---|---|
| Study Week | 2001133 | | 2001134 | | |
| Blinded Phase | N | % | N | % | Totals |
| 0 (Enrollment) | 526 | 100% | 533 | 100% | 1,059 |
| 24 (Completion) | 451 | 86% | 418 | 78% | 869 (82%) |
| Open Label | | | | | |
| Phase | | | | | |
| 24 (Enrollment) | 449 | 100% | 388 | 100% | 837 |
| 52 (Completion) | 380 | 85% | 261 | 67% | 641 (77%) |

### Demographic Data—Combined Trials

Demographics are similar across between the placebo and TTS groups. In the 4 trials combined (Phase 2b and 3 studies) the following baseline demographic and anthropometric data are observed among the 1,401 subjects:

1. Age: mean 49 (7.5 SD); median 49; Min-Max 26-70

2. Age distribution: 10% are 30-39; 41% are 40-49, 41% are 50-59; 6% 60-64

3. Race: 89% Caucasian; 6% African American; 3% Hispanic

4. Geography: 89% U.S.; 3% Canada; 4% Europe; 4% Australia

5. Marital status: 87% married to partner; 13% not married

6. Duration of relationship with partner (years): mean 18.6 (11.35 SD); median 17; Min-Max 1-54

7. Baseline weight (kg): mean 73.1 (15.3 SD); median 71; Min-Max 40.6-153.6

8. Baseline height (cm): mean 163.8 (6.38); median 163; Min-Max 132-183

9. Body Mass Index: mean 27.2 (5.51); median 26; Min-Max 17-58

10. Estrogen therapy route: 77% oral; 23% transdermal

11. Age at hysterectomy: mean 39.6 (7.9); median 40; Min-Max 17-66

12. Age at oophorectomy: mean 40.4 (7.8); median 41; Min-Max 17-66

13. Years since surgery: ˜9 (7.2); median 7-8; Min-Max 0.5-42.5

14. Tobacco use: 53% never; 30% previously; 17% currently

15. Alcohol use: 22% never; 4% previously; 73% currently

Mean scores at baseline: The overall mean scores at baseline in the two phase 2 and two phase 3 studies for the SAL 4-week frequency of total satisfying sexual events (3.0), PFSF sexual desire (21.2), and personal distress (64.5) are consistent with mean scores from other studies with women who considered themselves to have few satisfying sexual activities, low sexual desire, and are distressed about their lack of sexual desire. Baseline disease severity as judged by the three main endpoints (satisfying sexual events, distress scores, and desire scores) is similar between the active and placebo treatment groups across the four studies.

Estrogen formulations used: Overall, 77% of the subjects in the combined studies are taking concomitant oral estrogen therapy (ET) and 23% are receiving concomitant transdermal ET. Subjects who participate in Studies 2001133 and 2001134 use either oral or transdermal ET. Subjects in Study 1999068 use concomitant oral ET, whereas subjects in Study 1999092 use concomitant transdermal ET only. Of the 1,075 subjects on oral ET, the percentage of subjects taking higher dose oral ET (defined as >0.625 mg Premarin or its equivalent) is 48% and those on higher dose transdermal ET (defined as >0.05 mg) is 54%. The remaining subjects are receiving lower dose oral or transdermal ET. In the oral estrogen therapy group, the percentage of subjects taking conjugated equine estrogen (63%) and non-conjugated equine estrogen (37%) is also similar between the two treatment groups in the 3 studies using oral ET. The next most commonly used form of concomitant ET is estradiol.

### Primary Efficacy Outcome (Total SSEs at Week 24)

The primary endpoint is the change in SSEs (sum of the SAL Question 3 and 6—satisfying sexual activity with or without intercourse).

A discussion and analysis of the efficacy results follows, with results from the 4 individual studies shown first, followed by Figures (bar graphs) of the individual and combined results shown together.

TABLE 5
Number of Satisfactory Sexual Events

| | | | | | |
|---|---|---|---|---|---|
| Study Number | Testosterone | | Mean Change | Efficacy | |
| (Number of | dose | Baseline | from Baseline | Analysis | Results |
| subjects) | (mcg/day) | Mean^{a} | at Week 24^{a, b} | Method | p-value |

Frequency of Total Satisfying Sexual Episodes (SSEs)
Phase 3 trials

| | | | | | |
|---|---|---|---|---|---|
| 2001133 | 0 (placebo) | 2.94 | 0.98 | ANCOVA | p = 0.0003 |
| (N = 562) | 300 | 2.82 | 2.13 | | |
| 2001134 | 0 (placebo) | 3.19 | 0.73 | Wilcoxon | p = 0.0010 |
| (N = 532) | 300 | 3.04 | 1.56 | Rank Sum^{b} | |

Phase 2 trials

| | | | | | |
|---|---|---|---|---|---|
| 1999068 | 0 (placebo) | 2.8 | 1.20 | Poisson | p = 0.0493 |
| (N = 229) | 300 | 2.92 | 2.32 | Regression | |
| 1999092 | 0 (placebo) | 3.20 | 1.12 | Poisson | p = 0.064l |
| (N = 76) | 300 | 2.08 | 3.08 | Regression | |

^{a}The frequency of SSEs is based on data from the SAL from baseline and the last 4 recorded weeks.
^{b}Mean change from baseline adjusted for age, estrogen therapy route, pooled centers, and baseline value. For frequency of total satisfying episodes in Study 2001134, the ANCOVA model assumption of normality is severely violated; therefore, unadjusted mean change from baseline (i.e. no covariate adjustment) is shown and Wilcoxon rank sum analysis is conducted.
The table above shows the mean change from baseline in the SSEs for the last recorded 4-week period of time. In both Phase 3 studies (2001133 and 2001134), compared with placebo, subjects treated with 300 mcg/day TTS have an increase in the frequency of total SSEs.

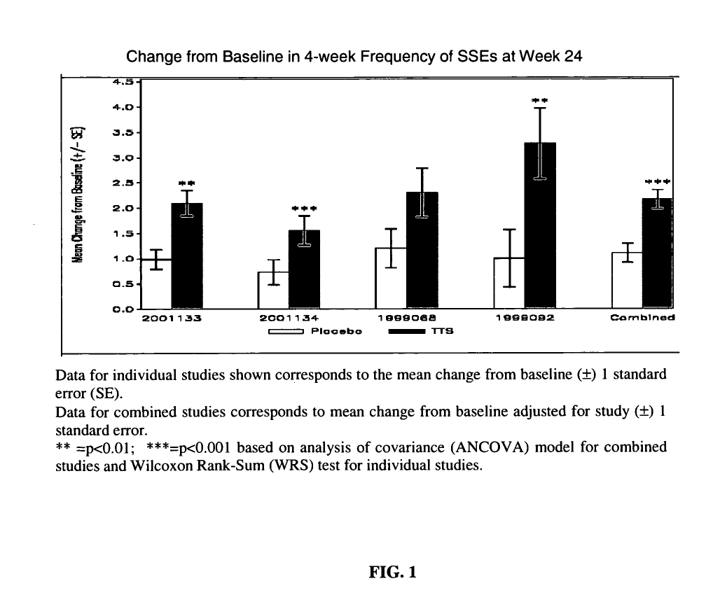

Mean changes from baseline on 300 mcg/day TTS and placebo are shown in FIG. 1.

### Change in Personal Distress

The PDS measures personal distress associated with subjects' low desire for, and lack of interest in, sex. The PDS instrument contains 9 items in a single domain. Subjects fill out the questionnaire based on their experiences and feelings over the preceding 30 days. A decrease in the PDS score indicates a decrease in a patient's distress.

Across the two Phase 3 trials, the baseline score for distress ranges from 62.57 to 66.38 for the placebo group and from 64.78 to 66.61 for the TTS group, which corresponds to an average answer of “often” being distressed for the 9 questions (see Table 6). The Least-Squares Means difference between the change in placebo and TTS scores is approximately 7.3.

TABLE 6
Change in Personal Distress Scores

| | | | | | |
|---|---|---|---|---|---|
| | | | Mean Change | Efficacy | |
| | Testosterone | Baseline | from Baseline | Analysis | Results |
| Study Number | dose (mcg/day) | Mean^{a} | at Week 24^{a, b} | Method | p-value |

Personal Distress Score
Phase 3 studies

| | | | | | |
|---|---|---|---|---|---|
| 2001133 | 0 | 62.57 | −16.31 | ANCOVA | p = 0.0006 |
| (N = 562) | 300 | 64.78 | −23.55 | | |
| 2001134 | 0 | 66.38 | −18.27 | ANCOVA | p = 0.0091 |
| (N = 532) | 300 | 66.61 | −24.34 | | |

Phase 2 trials

| | | | | | |
|---|---|---|---|---|---|
| 1999068* | 0 (placebo) | 64.71 | −18.46 | ANCOVA | p = 0.1258 |
| (N = 229) | 300 | 65.39 | −23.76 | | |
| 1999092* | 0 (placebo) | 56.73 | −2.11 | ANCOVA | p = 0.0025 |
| (N = 76) | 300 | 57.14 | −22.10 | | |

^{a}Range for distress score is 0-100.
^{b}Mean change from baseline adjusted for age, estrogen therapy route, pooled centers, and baseline value.

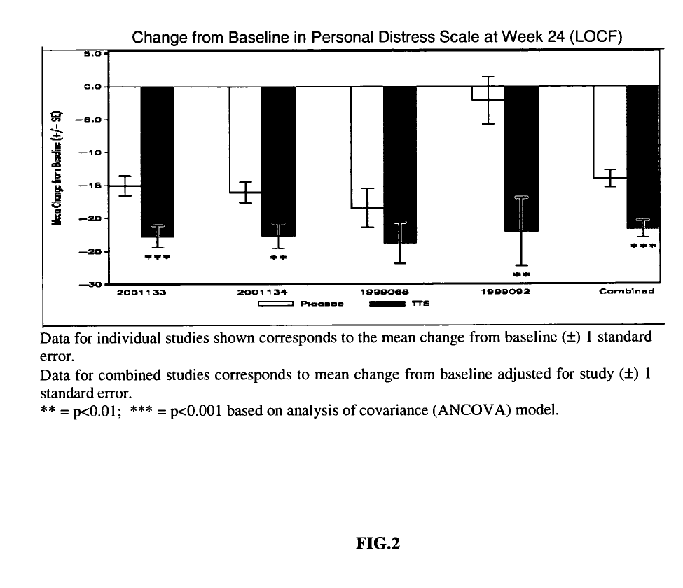

The changes from baseline in the PDS score at Week 24 for the four individual studies and the studies combined are shown in FIG. 2.

### Change in Sexual Desire

Change in desire is assessed using the Desire domain of the PFSF. Subjects are instructed that feelings could be either mental or physical and could occur in the absence of sexual activity. Their responses are to reference the past 30 days.

Across the two phase 3 trials, the baseline score for sexual desire ranges from 20.82 to 23.37 for the placebo group and from 19.79 to 21.67 for the active treatment group (see Table 7). This generally corresponds with an average answer of “seldom” for the 9 items in the desire domain.

TABLE 7
Change in Sexual Desire Score

| | | | | | |
|---|---|---|---|---|---|
| | | | Mean Change | Efficacy | |
| | Testosterone | Baseline | from Baseline | Analysis | Results |
| Study Number | dose (mcg/day) | Mean^{a} | at Week 24^{a, b} | Method | p-value |

Phase 3 trials

| | | | | | |
|---|---|---|---|---|---|
| 2001133 | 0 (placebo) | 20.82 | 6.90 | ANCOVA | p = 0.0006 |
| (N = 562) | 300 | 19.79 | 11.85 | | |
| 2001134 | 0 (placebo) | 23.37 | 6.21 | ANCOVA | p = 0.0006 |
| (N = 532) | 300 | 21.67 | 11.38 | | |

Phase 2 trials

| | | | | | |
|---|---|---|---|---|---|
| 1999068* | 0 (placebo) | 20.14 | 8.38 | ANCOVA | p = 0.0498 |
| (N = 229) | 300 | 20.94 | 13.67 | | |
| 1999092* | 0 (placebo) | 19.89 | 5.98 | ANCOVA | p = 0.0214 |
| (N = 76) | 300 | 23.12 | 16.43 | | |

*Source: E-Table 5, ISE.
^{a}Range for sexual desire score is 0-100.
^{b}Mean change from baseline adjusted for age, estrogen therapy route, pooled centers, and baseline value.

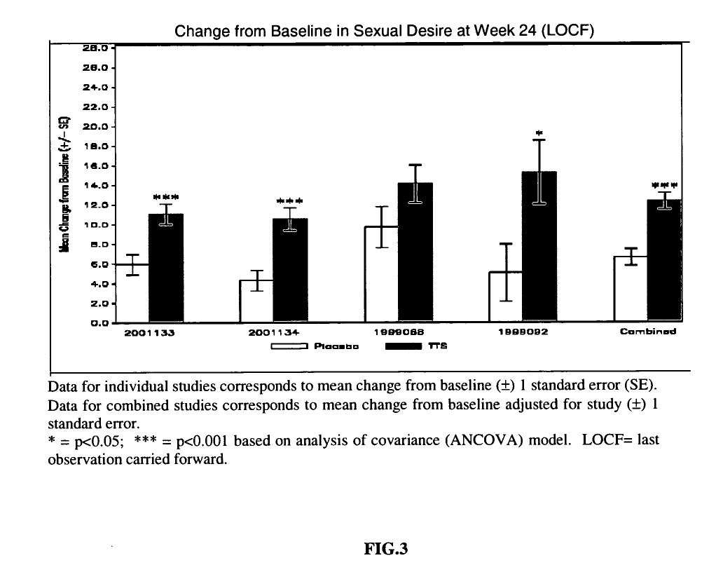

The changes from baseline in the PFSF Desire domain score at Week 24 for the four individual studies and the studies combined are shown in FIG. 3.

### Summary of Phase 3 Efficacy Findings

An overview of the results for the primary and two important secondary efficacy endpoints for the two phase 3 clinical trials can be found in Table 8.

TABLE 8
Changes in Frequency of SSEs, and Sexual Desire and Personal Distress Scores

| | | | | | |
|---|---|---|---|---|---|
| | | | Mean Change | Efficacy | |
| Endpoint | Testosterone | Baseline | from Baseline | Analysis | Results |
| Study Number | dose (mcg/day) | Mean^{a} | at Week 24^{a, b} | Method | p-value |

Frequency of Total SSEs

| | | | | | |
|---|---|---|---|---|---|
| 2001133 | 0 | 2.94 | 0.98 | ANCOVA | p = 0.0003 |
| | 300 | 2.82 | 2.13 | | |
| 2001134 | 0 | 3.19 | 0.73 | Wilcoxon | p = 0.0010 |
| | 300 | 3.04 | 1.56 | Rank Sum^{b} | |

Sexual Desire Score [range 0 to 100]

| | | | | | |
|---|---|---|---|---|---|
| 2001133 | 0 | 20.82 | 6.90 | ANCOVA | p = 0.0006 |
| | 300 | 19.79 | 11.85 | | |
| 2001134 | 0 | 23.37 | 6.21 | ANCOVA | p = 0.0006 |
| | 300 | 21.67 | 11.38 | | |

Personal Distress Score [range 0 to 100]

| | | | | | |
|---|---|---|---|---|---|
| 2001133 | 0 | 62.57 | −16.31 | | |
| | 300 | 64.78 | −23.55 | ANCOVA | p = 0.0006 |
| 2001134 | 0 | 66.38 | −18.27 | | |
| | 300 | 66.61 | −24.34 | ANCOVA | p = 0.0006 |

^{a}For frequency of total satisfying sexual episodes, baseline mean and mean change from baseline at Week 24 refer to the average 4-week frequency.
^{b}Mean change from baseline adjusted for age, estrogen therapy route, pooled centers, and baseline value. For frequency of total satisfying episodes in Study 2001134, the ANCOVA model assumption of normality is severely violated; therefore, unadjusted mean change from baseline (i.e. no covariate adjustment) is shown and Wilcoxon rank sum analysis is conducted.

### Pharmacokinetics of Free and Total Testosterone

For a population of reproductive aged women (ages 18-49 years), a reference range for free testosterone in this population is 0.9-7.3 pg/ml, and a reference range for total testosterone is 12-50 ng/dl.

The serum concentrations of free and total testosterone in participants on testosterone in Studies 133 and 134 who have blood samples collected within 5 days of applying the last patch prior to the scheduled study visit are presented in Table 9. While mean concentrations of total testosterone exceed the upper limit of normal for reproductive-aged women at all treatment visits, mean concentrations of free testosterone remain at the upper of the normal range throughout treatment.

TABLE 9
Studies 133 & 134: Free and Total Testosterone Concentrations

| Analyte | | | | | |
|---|---|---|---|---|---|
| Statistic | Week 0 | Week 12^{a} | Week 24 | Week 52 | Week 78 |
| Free Testos- | | | | | |
| terone (pg/mL) | | | | | |
| n | 544 | 209 | 412 | 287 | 91 |
| Mean | 0.92 | 5.52 | 4.36 | 4.44 | 5.94 |
| SE | 0.03 | 0.41 | 0.16 | 0.31 | 0.47 |
| Median | 0.80 | 4.00 | 3.60 | 3.10 | 4.60 |
| Min | 0.0 | 0.3 | 0.1 | 0.3 | 0.5 |
| Max | 8.8 | 61.8 | 30.4 | 63.1 | 25.1 |
| P-value* | | | | | 0.3917 |
| Total Testos- | | | | | |
| terone (ng/dL) | | | | | |
| n | 547 | 210 | 413 | 288 | 91 |
| Mean | 17.6 | 90.3 | 79.7 | 74.8 | 91.1 |
| SE | 0.4 | 4.4 | 2.7 | 3.6 | 6.0 |
| Median | 15.0 | 72.5 | 69.0 | 62.0 | 83.0 |
| Min | 0 | 7 | 3 | 6 | 14 |
| Max | 119 | 511 | 516 | 461 | 342 |
| P-value* | | | | | 0.0510 |

Data summarized in this table are for the subset of patients who had blood samples collected within 5 days of applying the last patch before scheduled study visit at timepoint.
n = number of patients with available data at timepoint;
SE = standard error;
TTS = 300 mcg/day testosterone transdermal system.
*Test of no effect over time in the log-transformed hormone concentration values across post-baseline visits for free, total, and bioavailablc testosterone and total dihydrotestosterone, and across all visits for sex hormone binding globulin, using repeated measures analysis of variance (ANOVA) adjusted by study and visit.
^{a}Total dihydrotestosterone was not collected at Week 12 in either study, according to the protocol.

Subgroup Analyses

Analysis of Frequency of Total Satisfying Episodes by ERT Group is shown for Surgically Menopausal women (for the four studies) in Table 10.

TABLE 10

| | Efficacy Result | | Hormone Summary | | | |
|---|---|---|---|---|---|---|
| | Difference | | TTS: Free T | | Baseline | |
| ERT | TTS versus | TTS-Change | at Week 24 | | SHBG | |
| Group | Placebo | from Baseline | Mean | Median | Mean | Median |
| Trans- | 2.14 | 3.00 | 6.0 | 5.4 | 51 | 48 |
| dermal* | | | | | | |
| Oral | 0.74 | 1.76 | 4.1 | 3.0 | 110 | 99 |
| CEE** | 0.44 | 1.41 | 3.6 | 2.7 | 119 | 108 |
| Non- | 1.17 | 2.31 | 5.0 | 3.7 | 95 | 84 |
| CEE | | | | | | |

*Statistically significantly greater treatment effect seen in Transdermal as compared to Oral p < 0.01).
**Treatment effect for CEE patients is not statistically significantly different from non-CEE patients (p = 0.16)

Analysis of Frequency of Total Satisfying Episodes by Oral ERT Subgroup for Naturally Menopausal woman in a separate clinical study (Study 2002006) is shown in Table 11:

TABLE 11

| | Efficacy Result | | Hormone Summary | | | |
|---|---|---|---|---|---|---|
| | Difference: | | TTS: Free T | | Baseline | |
| ERT | TTS versus | TTS-Change | at Week 24 | | SHBG | |
| Group | Placebo | from Baseline | Mean | Median | Mean | Median |
| Oral | 1.35 | 1.89 | 3.3 | 2.7 | 95 | 87 |
| CEE* | 0.93 | 1.64 | 2.8 | 2.3 | 110 | 100 |

*Treatment effect for CEE patients is not statistically significantly different from non-CEE patients (p = 0.13)

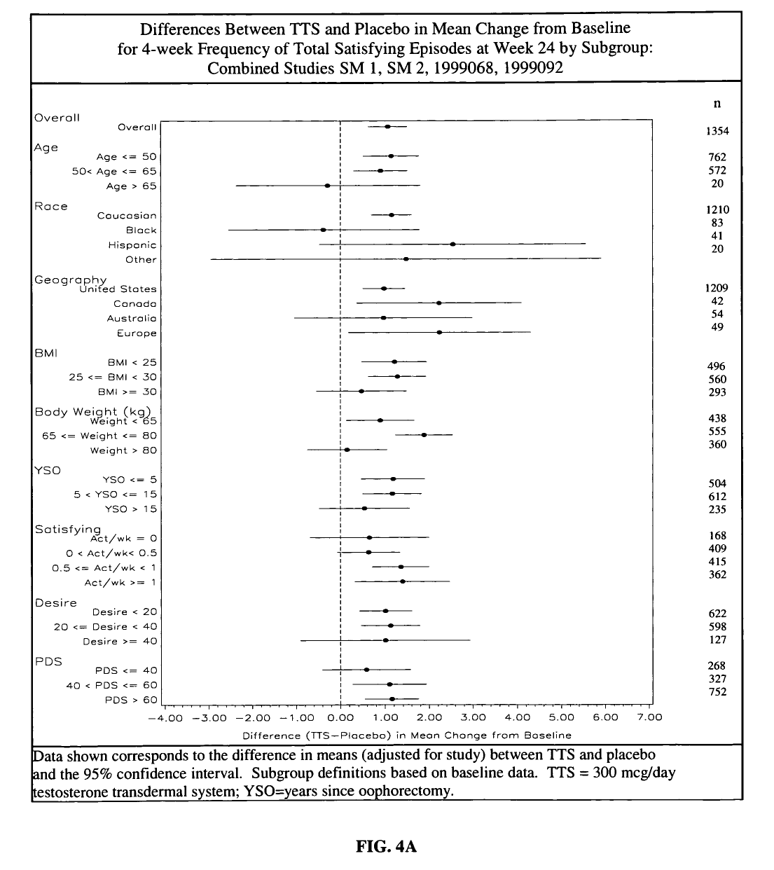

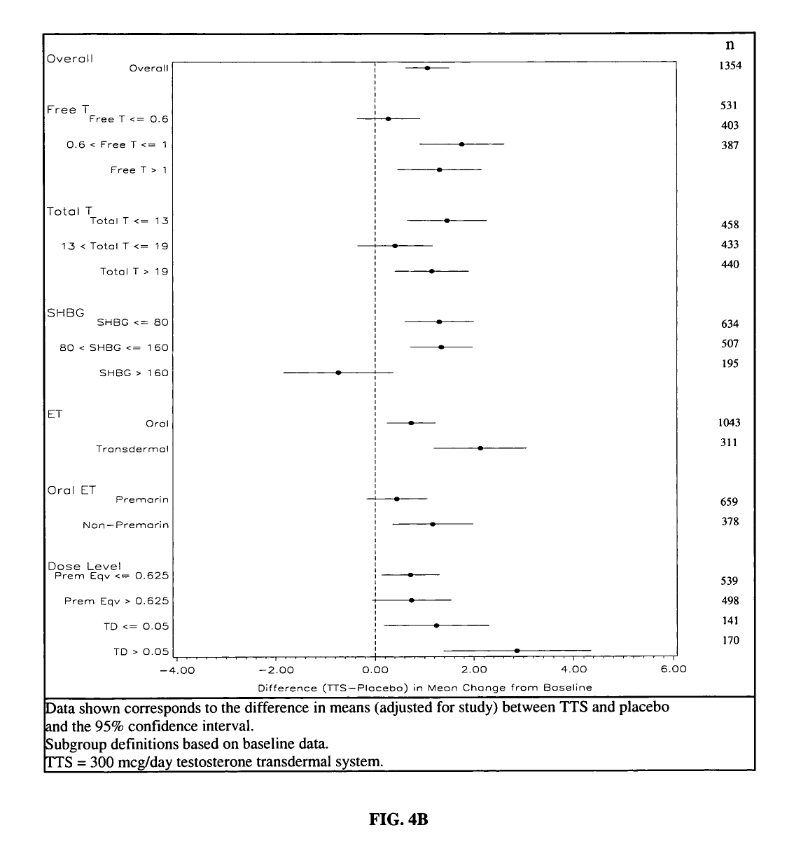

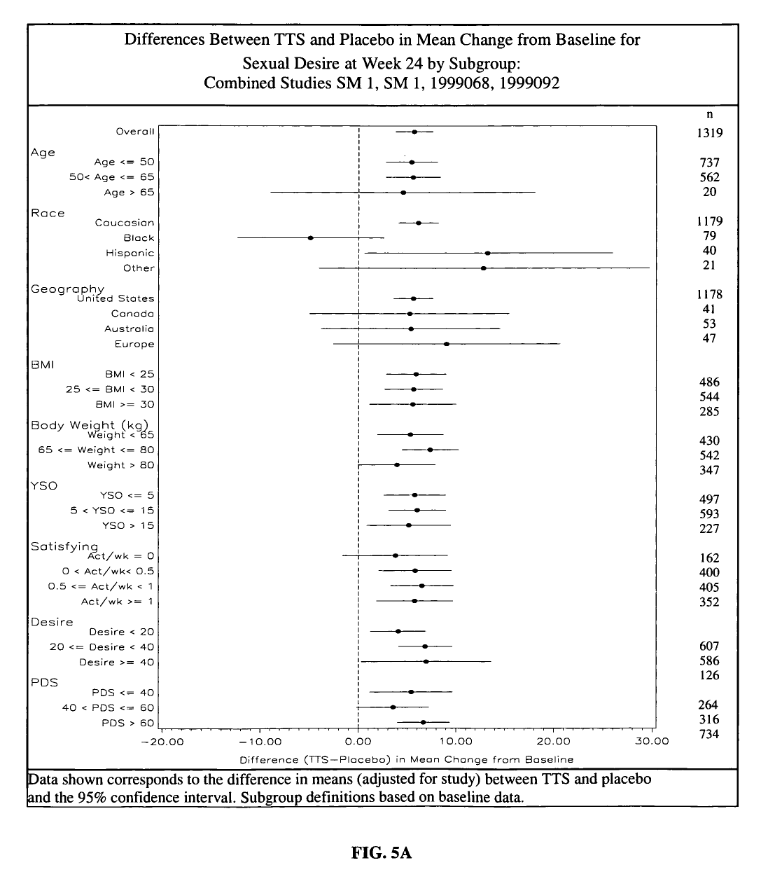

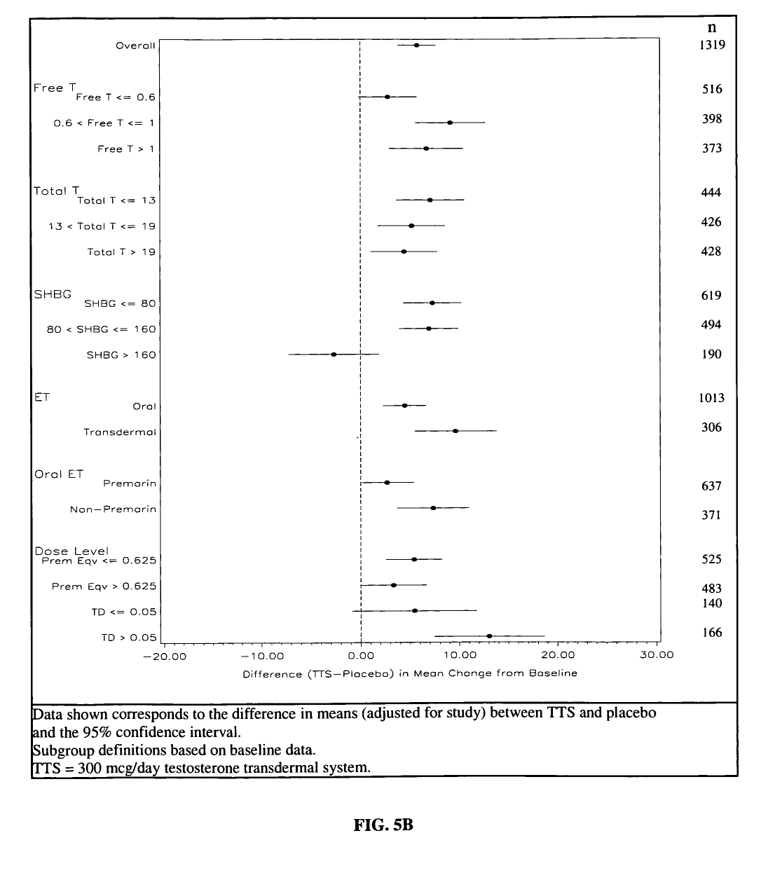

Efficacy is examined in a number of subpopulations defined on the basis of demographics and other characteristics at baseline (FIGS. 4A-4B and FIGS. 5A-5B).

Subgroup analyses of the combined Phase IIb and Phase III studies in surgically menopausal women support the effectiveness of TTS across a range of demographic and reproductive characteristics, disease severity, types of estrogen therapy, and baseline hormone concentrations. A generally consistent treatment effect across subpopulations is apparent in both total satisfying episodes and sexual desire.

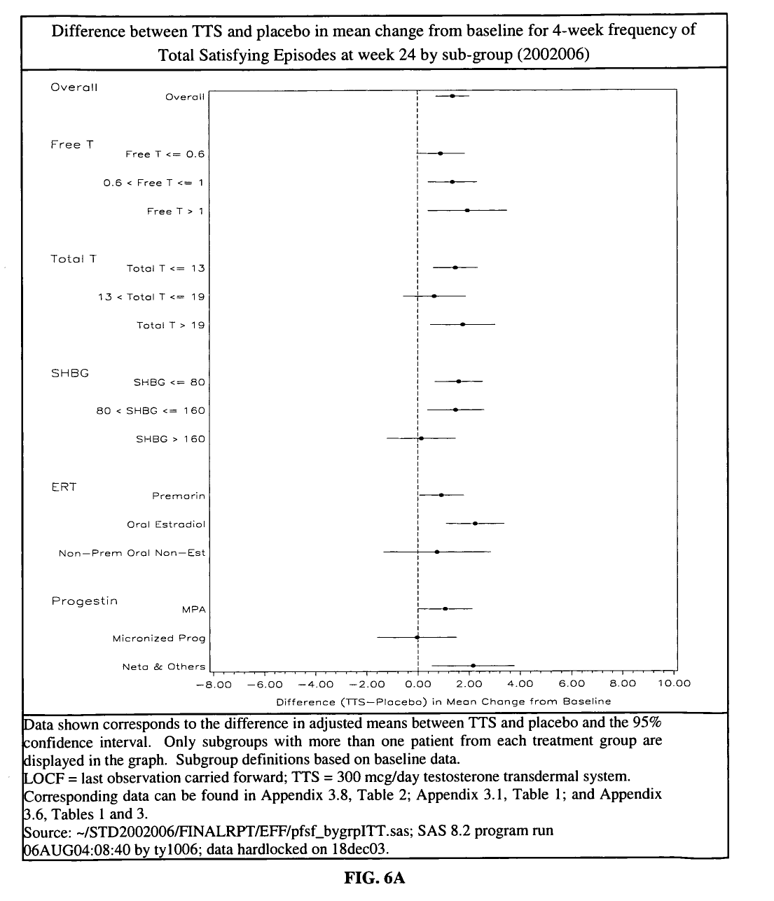

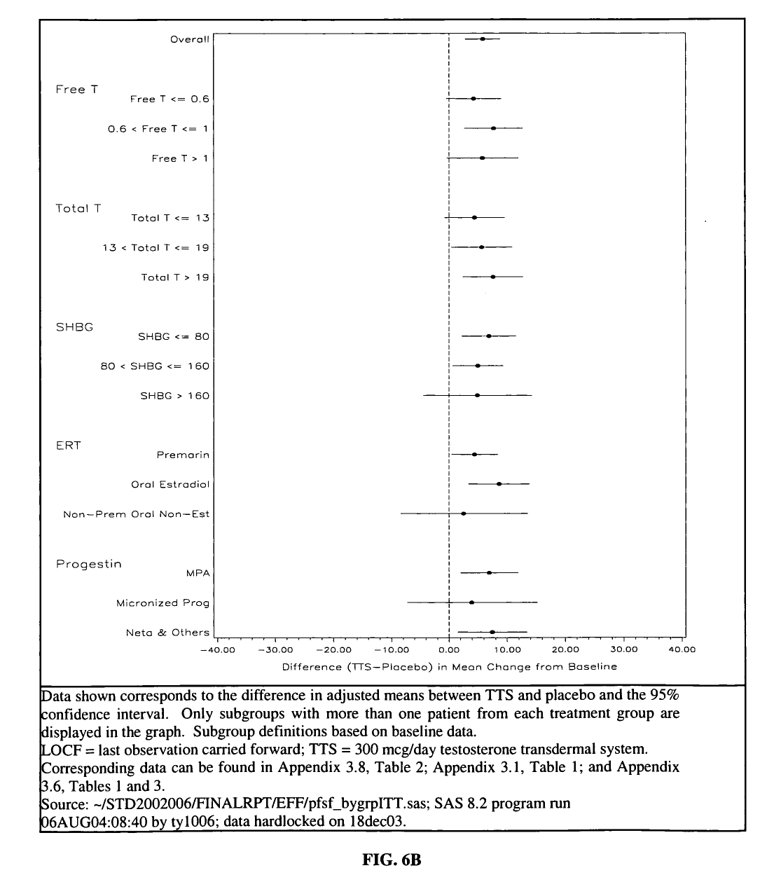

The magnitude of the treatment effect may be influenced by SHBG level, consistent with the fact that higher SHBG levels may result in lower free testosterone levels. When patient are subdivided by route of administration of concomitant estrogen, a larger treatment effect is seen in patients using transdermal estrogen. This finding may, in part, be attributed to effects of SHBG on free testosterone, as SHBG levels differ in patients on oral and transdermal estrogen. When users of oral estrogen products are further divided by type of estrogen [conjugated equine estrogens (CEE) v. non-CEE], patients using CEE tend to have lower efficacy than patients using other oral estrogens. The reasons for this difference may be in part a result of higher SHBG levels induced by CEE treatment, however, without being bound by theory, it is now believed that this may also be in part attributable to the type and route of administration of estrogen. Similarly, without being bound by theory, it is now believed that this may also be in part attributable to the type and route of administration of estrogen in naturally menopausal women (See FIGS. 6A-6B and Tables 19-22).

TABLE 12
Mean Weekly Frequency of Total Satisfying Episodes at Week 24 (LOCF)
Individual Studies 1999092, 1999068
(Phase IIb Intent-to-treat Patients)

| Study | Placebo | | | 300 mcg/day | | | Comparison with Placebo | | |
|---|---|---|---|---|---|---|---|---|---|
| Concomitant | | Adj. | | | Adj. | | Ratio of | | |
| ET | n | Mean | (95% CI) | n | Mean | (95% CI) | Rate^{a} | 95% CI | p-value^{b} |
| 1999092 | 36 | 0.94 | (0.68, 1.31) | 33 | 1.35 | (0.99, 1.84) | 1.43 | (0.98, | 0.0641 |
| Transdermal | | | | | | | | 2.09) | |
| ET | | | | | | | | | |
| 1999068 | 112 | 0.97 | (0.76, 1.23) | 108 | 1.26 | (1.03, 1.54) | 1.30 | (1.00, | 0.0493 |
| Oral ET | | | | | | | | 1.69) | |
| CEE | 73 | 0.90 | (0.73, 1.12) | 67 | 1.04 | (0.81, 1.34) | 1.16 | (0.85, | 0.3458 |
| | | | | | | | | 1.57) | |
| Non-CEE | 39 | 1.00 | (0.59, 1.72) | 38 | 1.42 | (1.01, 1.98) | 1.41 | (0.89, | 0.1462 |
| | | | | | | | | 2.25) | |

^{a}Ratio of rates corresponds to the ratio of weekly rate of the 300 mcg/day group to placebo, adjusted for baseline rate, age, marital status, and pooled site.
^{b}p-value corresponds to the test of the null hypothesis that the rate ratio is 1.
n = number of patients with responses;
Adj. Mean = mean weekly frequency adjusted for baseline rate, age, marital status, and pooled site;
CI = confidence interval.
ET = estrogen therapy

TABLE 13
Surgical Menopause Studies (2001133, 2001134, 1999068, 1999092)
4-week Frequency of Total Satisfying Activity by Estrogen Group

| | | | Hormone Summary-TTS Patients | | | |
|---|---|---|---|---|---|---|
| | Efficacy Result | | Free T at | | | |
| | | Treatment Effect: | Week 24 | | Baseline SHBG | |
| | | Difference in Means | Mean | Median | Mean | Median |
| Estrogen Group | N | TTS-Placebo (95% CI) | (pg/mL) | (pg/mL) | (nmol/L) | (nmol/L) |
| Transdermal* | 311 | 2.14⁺(1.19, 3.08) | 6.0 | 5.4 | 51 | 48 |
| Oral | 1043 | 0.74⁺(0.24, 1.23) | 4.1 | 3.0 | 110 | 99 |
| CEE** | 659 | 0.44 (−0.17, 1.06) | 3.6 | 2.7 | 119 | 108 |
| Non-CEE | 378 | 1.17⁺(0.35, 1.98) | 5.0 | 3.7 | 95 | 84 |

⁺Statistically significant treatment effect vs. placebo p ≦ 0.05
*Significantly different treatment effect by ET route (oral/transdermal) (p < 0.01)
**No significant difference in treatment effect by oral ET therapy (CEE/non-CEE) (p = 0.16)

TABLE 14
Surgical Menopause Studies (2001133, 2001134, 1999068, 1999092)
Sexual Desire and Distress by Estrogen Group

| | Efficacy Result | | | |
|---|---|---|---|---|
| | Sexual Desire | | PDS (Personal Distress Scale) | |
| | | Treatment Effect: | | Treatment Effect: |
| Estrogen | | Difference in Means | | Difference in Means |
| Group | N | TTS-Placebo (95% CI) | N | TTS-Placebo (95% CI) |
| Trans- | 306 | 9.64⁺ (5.45, 13.82) | 305 | −11.58⁺ (−17.71, |
| dermal* | | | | −5.46) |
| Oral | 1013 | 4.44⁺ (2.28, 6.61) | 1008 | −6.29⁺ (−9.69, −2.88) |
| CEE** | 637 | 2.67 (−0.01, 5.36) | 634 | −3.81 (−8.11, 0.50) |
| Non-CEE | 371 | 7.34⁺ (3.68, 10.99) | 369 | −10.35⁺ (−15.98, |
| | | | | −4.71) |

⁺Statistically significant treatment effect vs. placebo p ≦ 0.05
Desire: *Significantly different treatment effect by ET route (oral/transdermal) (p = 0.03)
**Significantly different treatment effect by oral ET therapy (CEE/non-CEE) (p = 0.04)
PDS: No significant difference in treatment effect by ET route (oral/transdermal) (p = 0.14).
No significant difference in treatment effect by oral ET therapy (CEE/non-CEE) (p = 0.07).

TABLE 15
Study 2004031 in Naturally and Surgically Menopausal Women Not On Estrogen or Estrogen/Progestin
Analysis of Change from Baseline in 4-week Frequency of Total Satisfying Activity at Week 24 (LOCF)
(Intent-to-treat Patients)

| | | Baseline^{a} | Week 24 (LOCF)^{a} | Change from Baseline | | | |
|---|---|---|---|---|---|---|---|
| Treatment | n | Mean (SE) | Mean (SE) | Mean (SE)^{a} | Median (P25, P75) | Difference (CI)^{a} | p-value^{b} |
| Placebo | 265 | 2.49 (0.16) | 3.24 (0.22) | 0.73 (0.19) | 0.0 (−1.0, 2.0) | 1.40 (0.76, 2.03) | <.0001 |
| TTS 300 | 254 | 2.42 (0.17) | 4.50 (0.30) | 2.12 (0.26) | 1.0 (0.0, 3.8) | | |

The last observation carried forward (LOCF) analysis at Week 24 was based on responses recorded on the last weekly SAL and the preceding 3 weekly SALs.
Difference (CI) corresponds to the difference in means between TTS and placebo and the 95% confidence interval.
n = number of patients with responses;
SE = standard error;
P25 = 25^{th} percentile;
P75 =75 percentile;
TTS = testosterone transdermal system (mcg/day).
^{a}Winsorized mean (1%).
^{b}p-value corresponds to Koch's nonparametric ANCOVA adjusting for menopausal status.

TABLE 16
Study 2004031 in Naturally and Surgically Menopausal Women not on Estrogen or Estrogen/Progestin
Analysis of Change from Baseline in Sexual Desire at Week 24 (LOCF)
(Intent-to-treat Patients)

| | | Baseline^{a} | Week 24 (LOCF) | Change from Baseline | | | |
|---|---|---|---|---|---|---|---|
| Treatment | n | Mean (SE) | Mean (SE) | Mean (SE) | Median (P25, P75) | Difference (CI) | p-value^{a} |
| Placebo | 249 | 20.00 (0.86) | 26.65 (1.15) | 6.65 (0.98) | 4.4 (−2.2, 11.1) | 6.97 (3.85, 10.10) | <.0001 |
| TTS 300 | 232 | 19.07 (0.80) | 32.70 (1.38) | 13.63 (1.31) | 11.1 (0.0, 24.4) | | |

The last observation carried forward (LOCF) analysis at Week 24 was based on responses recorded on the last PFSF evaluated.
Difference (CI) corresponds to the difference in means between TTS and placebo and the 95% confidence interval.
n = number of patients with responses;
SE = standard error;
P25 = 25^{th} percentile;
P75 = 75^{th} percentile;
TTS = testosterone transdermal system (mcg/day).
^{a}p-value corresponds to an ANCOVA adjusting for menopausal status.

TABLE 17
Study 2004031 in Naturally and Surgically Menopausal Women not on Estrogen or Estrogen/Progestin
Analysis of Change from Baseline in Personal Distress Scale (PDS) at Week 24 (LOCF)
(Intent-to-treat Patients)

| | | Baseline^{a} | Week 24 (LOCF) | Change from Baseline | | | ANOVA |
|---|---|---|---|---|---|---|---|
| Treatment | n | Mean (SE) | Mean (SE) | Mean (SE) | Median (P25, P75) | Difference (CI) | p-value^{a} |
| Placebo | 250 | 65.77 (1.64) | 50.98 (1.95) | −14.79 (1.78) | −11.4 (−34.3, 2.9) | −11.19 (−16.54, −5.85) | <.0001 |
| TTS 300 | 229 | 64.91 (1.66) | 38.93 (2.09) | −25.98 (2.10) | −22.9 (−48.6, 0.0) | | |

TABLE 18
Hormone Summary - TTS Patients

| | |
|---|---|
| Free T at | Baseline |
| Week 24 | SHBG |

| Mean | Median | Mean | Median |
|---|---|---|---|
| (pg/mL) | (pg/mL) | (nmol/mL) | (nmol/L) |
| 7.1 | 6.4 | 44.6 | 41 |

Based on interim data and TTS 300

TABLE 19
Difference in Means (TTS-Placebo) in Satisfying Episodes by
ERT Type and SHBG Category (SM Phase II/III Studies)

| | ERT Type | | |
|---|---|---|---|
| SHBG category | CEE | Non-CEE | TD |
| <80 | 0.58 | 0.83 | 2.11 |
| 80-<160 | 1.15 | 1.24 | 2.92 |
| >=160 | −1.35 | 1.49 | NA |

TABLE 20
Difference in Means (TTS-Placebo) in Satisfying Episodes by ERT
Type and SHBG Category (SM Phase II/III Studies)

| | ERT Type | | | | | |
|---|---|---|---|---|---|---|
| | CEE | | Non-CEE | | TD | |
| | Diff in | | Diff in | | Diff in | |
| SHBG category | Means | TTS change | Means | TTS change | Means | TTS change |
| ≦80 | 0.58 | 2.24 | 0.83 | 2.61 | 2.11 | 2.79 |
| 80-≦160 | 1.15 | 1.64 | 1.24 | 2.09 | 2.92 | 3.60 |
| >160 | −1.35 | 0.04 | 1.49 | 1.81 | NA | |

TABLE 21
Difference in Means (TTS-Placebo) in Satisfying Episodes by ERT
Type and SHBG Category
(NM 2002006 Phase III Study)

| ERT Type | |
|---|---|
| CEE | Non-CEE |

| | Difference | TTS: | Difference | TTS: |
|---|---|---|---|---|
| SHBG category | in Means | Change | in Means | Change |
| <80 | 1.23 | 2.07 | 1.78 | 1.97 |
| 80-<160 | 1.09 | 1.76 | 2.36 | 2.82 |
| >=160 | 0.14 | 0.6 | NA | NA |

TABLE 22
Difference in Means (TTS-Placebo) in Satisfying Episodes by ERT
Type and SHBG Category
(NM 2002006 Phase III Study)

ERT Type

| SHBG category | CEE | Non-CEE |
|---|---|---|
| <80 | 1.23 | 1.78 |
| 80-<160 | 1.09 | 2.36 |
| >=160 | 0.14 | NA |

### Certain Methods

In an embodiment of the invention there is provided a method for administering testosterone to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→transdermal estrogen; and
(b) administering testosterone transdermally to the woman.

In an embodiment of the invention there is provided a method for increasing the efficacy of testosterone administered to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→transdermal estrogen; and
(b) administering testosterone transdermally to the woman.

In an embodiment of the invention there is provided a method for the treatment of hypoactive sexual desire disorder in surgically menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→transdermal estrogen; and
(b) administering testosterone transdermally to the woman.

In an embodiment of the invention there is provided a method for the treatment of hypoactive sexual desire disorder in naturally menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→transdermal estrogen; and
(b) administering testosterone transdermally to the woman.

In an embodiment of the invention there is provided a method for administering testosterone to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

In an embodiment of the invention there is provided a method for increasing the efficacy of testosterone administered to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

In an embodiment of the invention there is provided a method for the treatment of hypoactive sexual desire disorder in surgically menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

In an embodiment of the invention there is provided a method for the treatment of hypoactive sexual desire disorder in naturally menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

In an embodiment of the invention, in any one or more of the above-mentioned methods for the administration of testosterone or methods for increasing the efficacy of testosterone being administered, preferably the testosterone is being administered to the woman for the treatment of Female Sexual Dysfunction, more preferably for the treatment of Hypoactive Sexual Desire Disorder.

In an embodiment of the invention, in any one or more of the above-mentioned methods for the administration of testosterone or methods for increasing the efficacy of testosterone being administered preceding the paragraph before this one, preferably the testosterone is being administered to the woman for the treatment of vasomotor symptoms associated with menopause.

In an embodiment of the invention, in any one or more of the above-mentioned methods for the administration of testosterone or methods for increasing the efficacy of testosterone being administered, preferably the woman in need of therapy is naturally menopausal.

In an embodiment of the invention, in any one or more of the above-mentioned methods for the administration of testosterone or methods for increasing the efficacy of testosterone being administered, preferably the woman in need of therapy is surgically menopausal.

In an embodiment of the invention, in any one or more of the above-mentioned methods, preferably the woman is administered from about 50 to about 500 μg/day of testosterone, more preferably from about 200 to about 400 μg/day of testosterone. In an embodiment of the invention, in any one or more of the above-mentioned methods which precede this paragraph, preferably the woman is administered from about 100 to about 300 μg/day of testosterone or alternatively, from about 300 to about 500 μg/day of testosterone.

In an embodiment of the invention, in any one or more of the above-mentioned methods, preferably the estrogen is conjugated equine estrogen, more preferably the conjugated equine estrogen is selected from PREMARIN (conjugated estrogens, USP) and an alkali metal salt of 8,9-dehyrdroestrone-3-sulfate ester, more preferably the alkali metal salt is selected from sodium, potassium, and lithium.

In an embodiment of the invention, in any one or more of the above-mentioned methods preceding the paragraph before this one, preferably the estrogen is CENESTIN (synthetic conjugated estrogens, A).

In an embodiment of the invention, in any one or more of the above-mentioned methods wherein the estrogen is conjugated equine estrogen, preferably the amount of conjugated estrogen being administered is selected from the group consisting of: (a) from about 0.15 mg/day to about 0.25 mg/day; (b) from about 0.25 mg/day to about 0.5 mg/day; (c) from about 0.5 mg/day to about 0.625 mg/day; and (d) about 0.625 mg/day or greater.

In an embodiment of the invention, in any one or more of the above-mentioned methods, preferably wherein the transdermal administration of testosterone is via a patch, more preferably wherein the patch is selected from the group consisting of a matrix patch and a liquid reservoir patch, more preferably wherein the patch is a matrix patch.

In an embodiment of the invention, in any one or more of the above-mentioned methods preceding the paragraph before this one, preferably the transdermal administration of testosterone is via a gel, cream, spray, or ointment, or a combination thereof.

In an embodiment of the invention, in any one or more of the above-mentioned methods preceding the paragraph before this one, preferably the type and route of administration of the estrogen is oral conjugated equine estrogen and the another type and route of administration is oral conjugated non-equine estrogen. In an embodiment of the invention, in any one or more of the above-mentioned methods preceding the paragraph before this one, preferably the type and route of administration of the estrogen is oral conjugated equine estrogen and the another type and route of administration is oral non-conjugated estrogen. In an embodiment of the invention, in any one or more of the above-mentioned methods preceding the paragraph before this one, preferably the type and route of administration of the estrogen is oral conjugated equine estrogen and the another type and route of administration is transdermal estrogen. In an embodiment of the invention, in any one or more of the above-mentioned methods preceding the paragraph before this one, preferably the type and route of administration of the estrogen is oral conjugated equine estrogen and the another type and route of administration is no estrogen.

Unless context clearly dictates otherwise, the articles “a”, “an”, and “the” mean “one or more”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for administering testosterone to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

2. A method for increasing the efficacy of testosterone administered to a menopausal woman in need of receiving transdermal testosterone therapy wherein said woman is being coadministered estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

3. A method for the treatment of hypoactive sexual desire disorder in surgically menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

4. A method for the treatment of hypoactive sexual desire disorder in naturally menopausal women receiving concomitant estrogen, comprising the steps, in any order, of:
(a) determining the type and route of administration of the estrogen, then discontinuing administration of the estrogen and either simultaneously or subsequently beginning administration of estrogen to the woman by another type and route of administration, wherein said another type and route of administration is selected from the group of consisting of oral conjugated non-equine estrogen, oral non-conjugated estrogen, transdermal estrogen, and no estrogen; and wherein said another type and route of administration is listed at least one place to the right of the type and route of administration according to the following ordering: oral conjugated equine estrogen→oral conjugated non-equine estrogen→oral non-conjugated estrogen→no estrogen; and
(b) administering testosterone transdermally to the woman.

5. The method of claim 1, wherein testosterone is being administered to the woman for the treatment of Female Sexual Dysfunction.

6. The method of claim 1 wherein the woman in need of therapy is naturally menopausal.

7. The method of claim 1 wherein the woman in need of therapy is surgically menopausal.

8. The method of claim 1 wherein the woman is administered from about 50 to about 500 μg/day of testosterone.

9. The method of claim 8 wherein the woman is administered from about 100 to about 300 μg/day of testosterone.

10. The method of claim 8 wherein the woman is administered from about 200 to about 400 μg/day of testosterone.

11. The method of claim 8 wherein the woman is administered from about 300 to about 500 μg/day of testosterone.

12. The method of claim 1 wherein the estrogen is conjugated equine estrogen.

13. The method of claim 12 wherein the conjugated equine estrogen is PREMARIN (conjugated estrogens, USP).

14. The method of claim 12 wherein the conjugated equine estrogen comprises an alkali metal salt of 8,9-dehydroestrone-3-sulfate ester.

15. The method of claim 1 wherein the estrogen is CENESTIN (synthetic conjugated estrogens, A).

16. The method of claim 12 wherein the amount of conjugated estrogen being administered is from about 0.15 mg/day to about 0.25 mg/day.

17. The method of claim 12 wherein the amount of conjugated estrogen being administered is from about 0.25 mg/day to about 0.5 mg/day.

18. The method of claim 12 wherein the amount of conjugated estrogen being administered is from about 0.5 mg/day to about 0.625 mg/day.

19. The method of claim 12 wherein the amount of conjugated estrogen being administered is about 0.625 mg/day or greater.

20. The method of claim 1 wherein the transdermal administration of testosterone is via a patch.

21. The method of claim 1 wherein the transdermal administration of testosterone is via a gel, cream, spray, or ointment, or a combination thereof.

22. The method of claim 1 wherein the type and route of administration of the estrogen is oral conjugated equine estrogen and the another type and route of administration is oral conjugated non-equine estrogen.

23. The method of any one of claim 1 wherein the type and route of administration of the estrogen is oral conjugated equine estrogen and the another type and route of administration is oral non-conjugated estrogen.

24. The method of claim 1 wherein the type and route of administration of the estrogen is oral conjugated equine estrogen and the another type and route of administration is no estrogen.

25. The method of claim 1 wherein testosterone is being administered to the woman for the treatment of vasomotor symptoms associated with menopause.
